(19) United States
(12) Patent Application Publication
(10) Pub. No.: US 2004/0265874 A1
(43) Pub. Date: Dec. 30, 2004

(54) Pattern recognition method for diagnosis of systemic autoimmune diseases

(75) Inventors: Steven R. Binder, Berkeley, CA (US); John Glossenger, Benicia, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Bio-Rad Laboratories, Inc., Hercules, CA

(21) Appl. No.: 10/828,846

(22) Filed: Apr. 20, 2004

Related U.S. Application Data
(63) Continuation-in-part of application No. 09/691,405, filed on Oct. 17, 2000.

(51) Int. Cl. C12Q 1/68; G06F 19/00; G01N 33/48; G01N 33/50

(52) U.S. Cl. 435/6; 702/20

(57) ABSTRACT

An array of autoantibodies is quantitated in a patient sample and analyzed toward a diagnosis of systemic autoimmune diseases. The analysis uses any of various known pattern recognition techniques, for example k-nearest neighbor analysis, to compare the array of quantitation data to sets of data previously obtained from subjects having known systemic autoimmune diseases, thereby determining the particular disease(s) that the patient is suffering from as well as the degree of confidence or likelihood of accuracy of the determination. The method is effective in identifying a single disease and also in identifying two or more diseases simultaneously present. The method is readily susceptible to automated data processing, eliminating much of the human judgment and error that were previously entailed in diagnosing these diseases.

### CROSS-REFERENCES TO RELATED APPLICATIONS

This application is a continuation-in-part of U.S. application Ser. No. 09/691,405, filed Oct. 17, 2000, the contents of which are hereby incorporated by reference.

### BACKGROUND OF THE INVENTION

1. Field of the Invention

This invention relates to the field of autoimmune diseases and diagnostic methods for these and other diseases. The invention also relates to statistical methods of data analysis and their application to immunodiagnostics.

2. Description of the Prior Art

Autoimmune diseases are conditions in which the immune system attacks cells, tissues, and organs of one's own body rather than bacteria, viruses, and other microbes that invade the body from the outside. There are many different autoimmune diseases, attacking different parts of the body ranging from the gut to the brain, for example, or from the vascular system to the skin, and the attacks occur in different ways. Some autoimmune diseases are tissue- or organ-specific, while others affect several tissues. Diseases of this latter category are termed “systemic autoimmune diseases,” and the symptoms may vary from one patient to the next, with tissue injury and inflammation occurring in multiple sites in organs without relation to their antigenic makeup. Systemic autoimmune diseases are generally referred to as rheumatic diseases or connective tissue diseases (CTDs). Examples of systemic autoimmune diseases are rheumatoid arthritis, systemic lupus erythmatosus (SLE), scleroderma, polymyositis, dermatomyositis, Sjögren's syndrome (SS), and spondyloarthropathies such as ankylosing spondylitis. Autoimmune diseases are generally multifactorial in origin, with some of the contributing factors being genetic disposition, host factors (such as T cell defects and polyclonal stimulation of B cells that are resistant to controls), environmental factors (such as certain microbial infections), and antigen-driven mechanisms (such as sequestered antigens or cross-reacting exogenous antigens). Autoimmune diseases are still being identified (for example anti-phospholipid syndrome) and established diseases are frequently identified as autoimmune in nature (for example celiac disease).

Due to their overlapping symptoms and complex etiologies, autoimmune diseases are difficult to diagnose. Attempts at diagnosis are generally based on symptoms, together with the findings from a physical examination and results obtained from laboratory tests. Symptoms of many autoimmune diseases are nonspecific, and while laboratory test results may help they are often inadequate to confirm a diagnosis, particularly in the early phases of the disease. The problem is aggravated when the symptoms are transient and the laboratory results are inconclusive, as they are in many cases. For some autoimmune diseases, the patient will respond completely to treatment if the disease is identified at an early stage. Often, however, a specific diagnosis cannot be made until the disease is in an advanced state. The problem is particularly acute with systemic autoimmune diseases.

It is commonly believed that the presence of autoimmune antibody is indicative of an autoimmune disease. This belief is disproved however by the fact that almost all antibodies are present in measurable amounts in any individual, and comparisons with reference levels from healthy individuals are frustrated by the wide variations in normal antibody levels due to demographics such as gender, age, geographical region of domicile, nationality and race. In addition, the number of differentiable antibodies that must be investigated in the diagnosis is large, which presents an often unmanageable burden on the clinical laboratory in its attempts to obtain a specific and reasonably accurate diagnosis.

The most widely used method of identifying a systemic autoimmune disease is indirect immunofluorescence (IFA) microscopy, a manual method that requires a well trained technician. The result appears as a pattern of distinctive characteristics that are seen on cells (typically HeLa cells) that have been fixed on a slide, treated with serum, washed and then labeled. The distinctive characteristics of the pattern are a relatively speckled, relatively homogeneous appearance, or the like, and diagnosis is achieved by comparing the pattern with those of individuals with known diseases. The most common patterns are associated with several diseases, however, which obscures the diagnosis. As a result, additional testing for specific antibodies is needed. Even then, an experienced immunologist or rheumatologist must be called upon to interpret the results and make the final diagnosis.

Recently, enzyme-linked immunosorbent assays (ELISAs) have become available for preliminary screening of patients with symptoms suggesting an autoimmune disease. Specimens reading positive in the screening assay are then submitted for a specific diagnostic assay to determine which particular autoantibody is present and therefore which specific disease is suggested. Many autoimmune disease patients, however, suffer from two or more different autoimmune diseases, and this confounds efforts at achieving an accurate diagnosis when using this diagnostic method. Further, many physicians ordering screening tests (e.g., ANA screens) today have limited experience with specific antibody results. Results can be difficult to interpret. As a result, the utilization of specific antibody results is typically less than optimal. Moreover, the delay between the generation of a positive screen and the generation of the specific antibody results can be difficult both for the patient and for the physician, given the low positive predictive value of the initial result. When results are received, they may be highly suggestive in some cases (e.g., positive anti-Scl-70 antibody) but they are often not so useful (e.g., positive anti-SmRNP alone). Even extremely experienced rheumatologists may lack the ability to associate less common patterns with disease states.

It is therefore desirable to provide automated systems and methods that offer a useful indication of whether a patient may be suffering from a systemic autoimmune disease based on specific antibody test results. Such systems and methods should also indicate whether two or more such diseases are suspect.

### BRIEF SUMMARY OF THE INVENTION

The difficulties enumerated above and others are addressed by aspects of the present invention which provides systems and methods for identifying the presence of one or more systemic autoimmune diseases in a patient sample. In one aspect, multianalyte test data is obtained from a patient suspected of suffering from a systemic autoimmune disease, and a statistical pattern recognition process or algorithm is applied to this data to compare it with a multitude of reference data sets. The results of the comparison are used to provide an indication of the specific disease(s) that the patient may be suffering from. With the aid of computer software, the pattern recognition algorithm processes the entire pattern of results in a medical decision support system (MDSS). Applying pattern recognition processes in this manner permits a clinician to use test results from a single biological sample obtained from the patient, and to obtain both a diagnosis of the disease(s) and an assessment of the confidence level of the diagnosis. The MDSS results may be used by a clinician as a diagnostic assistance tool, e.g., to assist a clinician with making a diagnosis. A clinician may also use the MDSS results to suggest follow-up tests. The autoantibodies selected can be those that are known to be frequently elevated in various systemic autoimmune diseases, and particular antibodies can be included or omitted from the set in accordance with their perceived relevance to the particular disease or group of possible diseases. Two or more diseases can be diagnosed simultaneously, and confidence levels assigned to each, without the need for separate samples or analyses.

The present invention offers a number of advantages over diagnostic methods currently known. One advantage is the elimination of the need for a manual assay, since the multianalyte assay itself can be performed by automated instrumentation. Another is the ability to obtain a diagnosis without the need for screening followed by confirmatory tests. In addition, the invention provides a suggested diagnosis among a large number of possible diseases with a single-step sample analysis. Further, the invention eliminates the need for the intervention of a skilled professional to obtain an interpretation of the result. The result can therefore be transmitted directly to the ordering physician, even if that physician is a generalist and unfamiliar with the autoantibodies being measured.

According to one aspect of the present invention, a computer implemented method is provided for identifying whether a patient test sample is associated with one or more of a plurality of specific systemic autoimmune diseases (SADs) based on autoantibody levels present in the patient test sample. The method typically includes storing a plurality of reference data sets in a memory, each data set having values representing levels for each of a plurality of specific autoantibodies, wherein the reference data sets include, for each of the specific SADs, at least one reference data set having an association with the specific SAD, and wherein the reference data sets include at least one reference data set associated with none of the specific SADs. The method also typically includes receiving a sample data set having values representing levels for each of said plurality of autoantibodies for a patient test sample, and automatically applying a k-nearest neighbor process to the sample data set and the reference data sets to produce a statistically derived decision indicating whether the patient test sample is associated with none, one or more of said specific SADs.

According to another aspect of the present invention, a computer system is provided that is configured to provide output data indicating whether a patient test sample is associated with one or more of a plurality of specific systemic autoimmune diseases (SADs) based on autoantibody levels present in the patient test sample. The system typically includes a storage means for storing a plurality of reference data sets, each data set having values representing levels for each of a plurality of specific autoantibodies, wherein the reference data sets include, for each of the specific SADs, at least one reference data set having an association with the specific SAD, and wherein the reference data sets include at least one reference data set associated with none of the specific SADs. The system also typically includes a means for receiving a sample data set having values representing levels for each of the plurality of autoantibodies for a patient test sample, and a means for processing the sample data set and the reference data sets using a k-nearest neighbor process to produce a statistically derived decision indicating whether the patient test sample is associated with none, one or more of the specific SADs. The system may further include a means for providing output data including the statistically derived decision.

These and other objects, advantages, features and embodiments of the invention will be apparent from the description that follows.

### BRIEF DESCRIPTION OF THE DRAWINGS

FIG. 1 illustrates a medical decision support system (MDSS) where the present invention might be implemented.

FIG. 2 illustrates an example of a data table according to the present invention.

FIGS. 3 and 4 illustrate an example of KNN process logic according to one embodiment of the present invention.

FIG. 5 illustrates an example of a GUI display pane shown by the MDSS system.

FIG. 6 illustrates a possible user display output generated by the MDSS system.

### DETAILED DESCRIPTION OF THE INVENTION

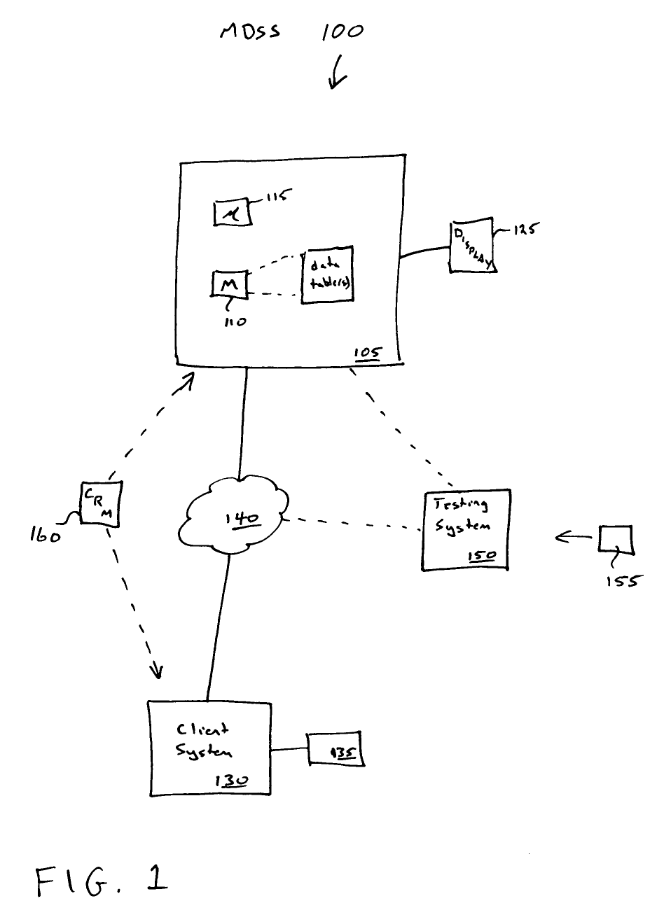

FIG. 1 illustrates a medical decision support system (MDSS) 100 where the present invention might be implemented. As shown, MDSS 100 includes MDSS computer system 105 having a processor 115 and memory module 110 as shown. Computer system 105 also includes communication modules (not shown) for transmitting and receiving information over one or more direct connections (e.g., USB, Firewire or other interface) and one or more network connections (e.g., including a modem or other network interface device). Memory module 110 may include internal memory devices and one or more external memory devices. Computer 105 also includes a display module, such as a monitor or printer. In one aspect, computer 105 receives data such as patient test results from test system 150, or other test result source, either through a direct connection or over a network 140. For example, test system 150 may be configured to run multianalyte tests on one or more samples 155 and automatically provide the test results to computer 105. Test system 150 may be directly coupled to computer system 105, or it may be remotely coupled over network 140. Computer 105 may also communicate data to and from one or more client systems 130 over network 140 as is well known. For example, a requesting physician may obtain and view a report from MDSS computer 105, which may be resident in a laboratory or hospital, using a client system 130.

Network 140 can be a LAN (local area network), WAN (wide area network), wireless network, point-to-point network, star network, token ring network, hub network, or other configuration. As the most common type of network in current use is a TCP/IP (Transfer Control Protocol and Internet Protocol) network such as the global internetwork of networks often referred to as the “Internet” with a capital “I,” that will be used in many of the examples herein, but it should be understood that the networks that the present invention might use are not so limited, although TCP/IP is the currently preferred protocol.

Several elements in the system shown in FIG. 1 may include conventional, well-known elements that need not be explained in detail here. For example, computer 105 could include a desktop personal computer, workstation, mainframe, laptop, etc. Each client system 130 could include a desktop personal computer, workstation, laptop, PDA, cell phone, or any WAP-enabled device or any other computing device capable of interfacing directly or indirectly to the Internet or other network connection. Client system 130 typically runs an HTTP client, e.g., a browsing program, such as Microsoft's Internet Explorer™ browser, Netscape's Navigator™ browser, Opera's browser, or a WAP-enabled browser in the case of a cell phone, PDA or other wireless device, or the like, allowing a user of client system 130 to access, process and view information and pages available to it from computer 105 over network 140. Each client system 130 also typically includes one or more user interface devices, such as a keyboard, a mouse, touch screen, pen or the like, for interacting with a graphical user interface (GUI) provided by the browser on a display (e.g., monitor screen, LCD display, etc.) 135 in conjunction with pages, forms and other information provided by computer 105. As discussed above, the present invention is suitable for use with the Internet, which refers to a specific global internetwork of networks. However, it should be understood that other networks can be used instead of the Internet, such as an intranet, an extranet, a virtual private network (VPN), a non-TCP/IP based network, any LAN or WAN or the like.

According to one embodiment, each client system 130 and all of its components are operator configurable using applications, such as a browser, including computer code run using a central processing unit such as an Intel Pentium processor or the like. Similarly, MDSS computer 105 and all of its components might be operator configurable using application(s) including computer code run using a central processing unit 115 such as an Intel Pentium processor or the like, or multiple processor units. Computer code for operating and configuring MDSS computer 105 to process data and test results as described herein is preferably downloaded and stored on a hard disk, but the entire program code, or portions thereof, may also be stored in any other volatile or non-volatile memory medium or device as is well known, such as a ROM or RAM, or provided on any other computer readable medium 160 capable of storing program code, such as a compact disk (CD) medium, digital versatile disk (DVD) medium, a floppy disk, and the like. Additionally, the entire program code, or portions thereof, may be transmitted and downloaded from a software source, e.g., over the Internet, or from a server, as is well known, or transmitted over any other conventional network connection as is well known (e.g., extranet, VPN, LAN, etc.) using any communication medium and protocols (e.g., TCP/IP, HTTP, HTTPS, Ethernet, etc.) as are well known. It will also be appreciated that computer code for implementing aspects of the present invention can be implemented in any programming language that can be executed on a computer system such as, for example, in C, C+, HTML, Java, JavaScript, or any other scripting language, such as VBScript.

In one preferred aspect of the present invention, MDSS computer 105 implements a pattern recognition system for analyzing patient test results against a database of stored test results, each associated with one or more diseases of known identity, or none. The data may be stored in one or more data tables or other logical data structures in memory 110 or in a separate storage or database system coupled with computer 105.

Pattern recognition systems for use in the practice of this invention typically begin with the development of a “training set,” a term understood in the art of statistical data analysis to mean data from a set of samples from reliable (“pedigreed”) sources. The set includes samples having disease conditions that are known from a previous and independent diagnosis as well as samples that are disease-free. A method in accordance with one aspect of the present invention thus begins with obtaining and analyzing reference samples from a series of subjects each of whom is known to have, or to have had, a particular systemic autoimmune disease, as well as a series of one or more subjects each of whom is known to be disease-free. This training set includes the full scope of the systemic autoimmune diseases sought to be investigated for a particular patient or for patients in general. Multianalyte analyses are performed on each sample in the training set, and the results are entered into the system and stored (e.g., in a database) in a manner resembling a multi-dimensional array in which each sample can be thought of as a point in a multi-dimensional space. The coordinates of the point that define its location in the space represent the value of the test results, one coordinate for each test. As a simple case, if the number of tests performed per sample in the training set is only two, the training set points can be visually arranged in a two-dimensional (x, y) plot, where the horizontal (x) axis corresponds to one of the tests and the vertical (y) axis corresponds to the other. In addition to its location in the two-dimensional plot (i.e., the x and y values) which is determined by the test results, each point is labeled as to the particular disease that is associated with its source. The corresponding array for a three-test system is a three-dimensional plot with orthogonal x, y, and z axes. Corresponding arrays for systems involving four or more tests are not capable of visualization, but are established in an analogous manner.

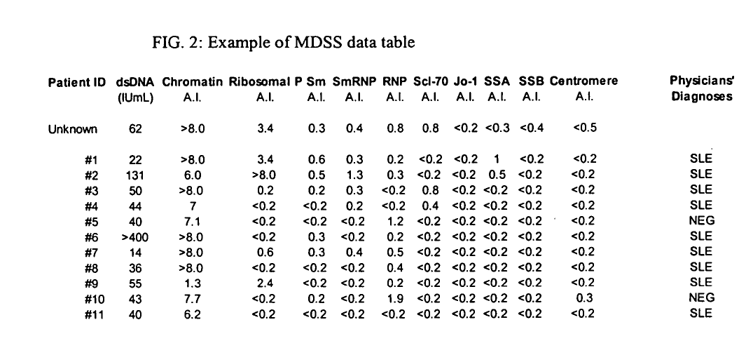

Once the training set is established, its test data is entered into the database. For example, the data can be stored in a database table logically arranged with columns or fields representing the results of antibody tests, or values representing the levels of antibodies. FIG. 2 illustrates an example of a portion of such a data table. As shown, an additional field may be provided to include a patient ID or other information identifying the origin of the sample. Demographic information, such as sex, age, etc. may also be included in fields. In this manner, each row (or record) of the table represents all the test values for a specific test sample. Each row also includes the previously diagnosed disease associated with the test sample, which diagnosis may be “none” or “NEG” if the patient sample is known to be disease free. In one embodiment, where a test set is associated with more than one disease, separate rows are created for each disease. For example, where one set of test data is associated with both SLE and SS, two rows are created in the training set data table, one for SLE and one for SS, with the values in the antibody fields being the same for each row.

Once the training data is entered into the database, the system is ready to receive data from a single patient sample or from a succession of patient samples with no need to recreate the database, or to re-analyze the training set, or to select and analyze a different training set. The patient sample is subjected to the same tests as the samples in the training set. An example for an “unknown” patient sample is shown in the top row in the table of FIG. 2. Using the illustrative representational description of the preceding paragraph, the patient sample test results may be visualized in an N-dimensional space as a point at a location defined by coordinates equal to the test values.

The determination of particular diseases is then achieved by a statistical comparison between the values of the patient sample test results (“unknown” in FIG. 2) and those of the training set. Various methods of statistical analysis can be used for the comparison. Examples are nearest neighbor (e.g., k-nearest neighbor) analysis, multi-linear regression analysis, Bayesian probabilistic reasoning, neural network analysis, and principal component analysis. While each of these methods is known in the art, the following explanations will assist the reader in understanding how the algorithm of each is applied in the practice of the present invention.

Nearest Neighbor Pattern Recognition

Nearest neighbor pattern recognition is a well established method for comparing different sets of data. It was widely used in the 1970's to automate analysis of mass spectral data, where the instrument output is a series of spectral lines (m/z ratios) and intensities. The user prepared a library of spectra of known compounds, or used a published library, and then used the nearest neighbor algorithm to evaluate unknowns. For example, the unknown might match against several phthalates in the library; perhaps none of them would be a perfect match or even a very good match, but the analyst would be confident that the unknown was a phthalate of some kind and this “clue” would be sufficient to identify the next steps required. The system of the present invention, in one aspect, operates in a similar manner. Since the input is a series of intensities (antibody levels), the intensities are compared against patients in a pre-established library, and the output are a series of possible matches to patients in the library. The system implements additional software processes to inspect the matches and produce an output that summarizes what disease state is most commonly observed in the patients with the best matches. The system also provides an output indicating a concordance value. For example, the output might include a concordance value or textual indicator concerning whether the disease state reported is “very strongly represented” amongst the patients with the best matches (e.g., 75% or more), or with a lesser degree of concordance, represented by “strong” and “moderate,” for example.

There are two important features in nearest neighbor pattern recognition. The first is the size and quality of the pre-established library (training set database) itself. The second is the number of neighbors that are collected for inspection. The number of neighbors is commonly referred to as “k”; hence the abbreviation kNN for k-Nearest Neighbor. These two features are linked, since a large library permits a larger value of k. In practice, it is preferable to use k values of 10-15, since this means that no match will ever be reported out because of a match to only one patient in the library. It will be appreciated that k values smaller than 10 or larger than 15 may be used. In one specific embodiment as will be discussed below, a k value of 11 is used.

In one k-nearest neighbor embodiment, the system reads the data from the patient sample into memory (e.g., memory 110) which may also contain the database of the training set. A process then calculates the numeric distances from the patient's test point to the data points in the memory (from the training set) that are in the vicinity of the patient's test point. From those points the process selects the k data points whose distances are the shortest or smallest. For example, numeric distance in two dimensions is typically a Cartesian distance metric of the form [(x₁−x₂)²+(y₁−y₂)²]^{1/2}. Thus, if k is 11, the algorithm selects the 11 data points having the shortest distances from the patient's test point. The disease associated with the k nearest data points is then identified as that which is present in the patient sample, and if the k points are associated with more than one disease, each of the diseases is indicated in the diagnosis. A refinement for the process is one in which the distance values of the various points are compared, and when the values for points representing two different diseases differ by less than a minimum difference, both diseases are considered to be equally likely. In a further refinement, the process is configured to process the numerical values of the distances to determine a confidence level. This can be done by using “similarity” values between the patient test results and each of the k data points and assigning a relative value to each disease by dividing the similarity for that disease by the sum of similarities. The final diagnosis is generally selected as that disease (or those diseases) with the highest relative value.

In general, the k-nearest neighbor algorithm is a “case-oriented” system that may be used to compare the patient's test data to the data from other individuals for whom demographic and other personal information is available in addition to the disease, such as age, sex, the length of time that the individual has had the disease, and similar factors. This information permits a diagnosing physician to exercise individual judgment as to whether or not to use the patients proposed by the process as reference points for the diagnosis.

Exemplary kNN Matching Process

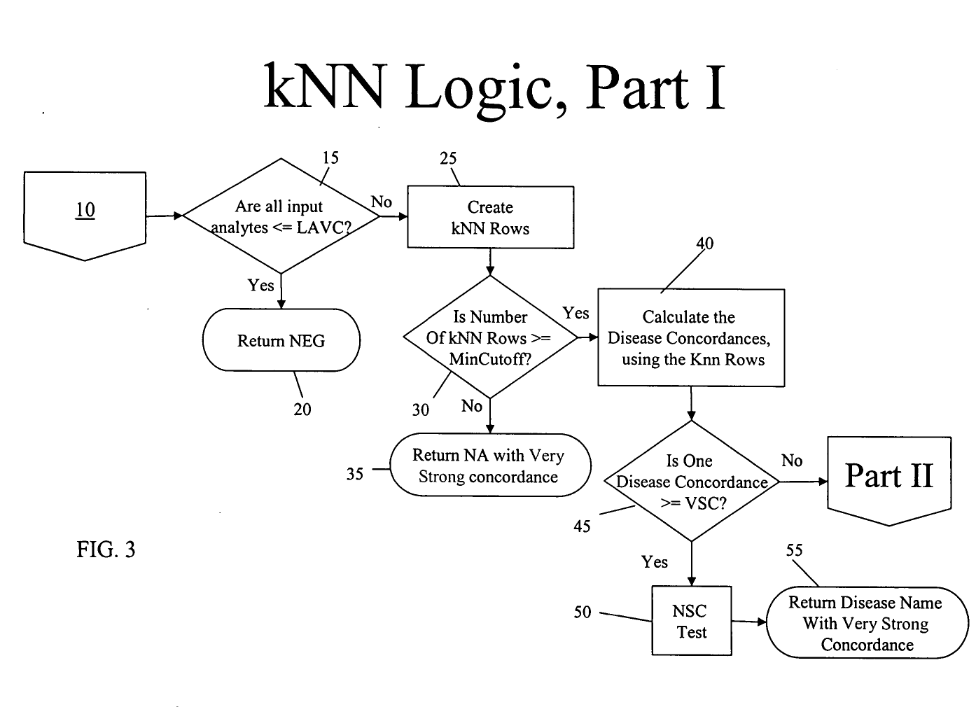

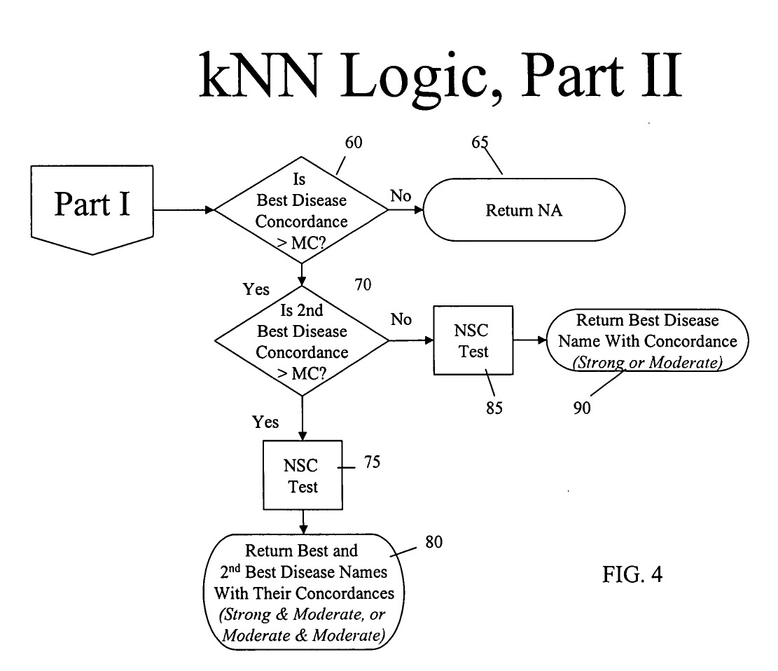

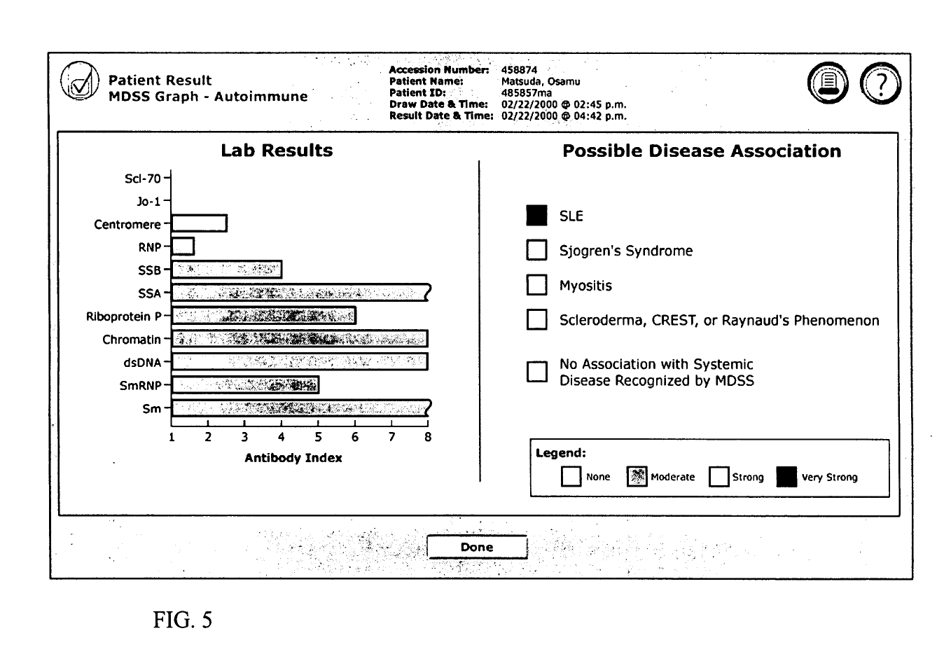

FIGS. 3 and 4 illustrate an exemplary KNN process according to one embodiment of the present invention. Briefly, the KNN algorithm executes in a process space (e.g., including one or multiple processors 105 and memory space) in MDSS 100. The KNN algorithm retrieves training set analyte values from the data table (e.g., via SQL calls), and processes the analyte values for the test sample using the retrieved data to produce an output. Preferably, the output is displayed on a screen (e.g., display monitor 125), printed, or sent to a remote system. The output, as will be described more later, may include, for example, a simple positive or negative indication, a diagnosis, or a graph including a diagnosis and rows returned from the database table, with suggested disease name and a row concordance for each row. For example, Table 1, below, is representative of a possible displayed output. Output data may also be provided to a client system 130 over network 140 for generation of a display on client monitor 135 or for further processing. FIG. 5 shows an example of one display output (e.g., pane displayed via browser) that shows a disease diagnosis, the strength of association between the patient test results and the diagnosed disease, and patient specific information such as patient ID. It should be appreciated that other display formats may be used and that other information may be displayed as desired.

As shown in FIG. 3, in optional process step 10, the input analyte values from the test subject or patient are normalized. For example, the values are converted into a normalized range from 0-100. In one embodiment, for example, analyte values are processed in antibody index format with the following calculation: log 10(x+1)·99.66. It should be appreciated that other normalization techniques may be used depending, in part, on the data format. In preferred aspects, the data stored in the data table is also normalized, however the table data may include data that is not normalized, in which case such data is preferably normalized when retrieved from the database. In step 15, the process determines whether all analyte input values meet a low analyte value cutoff (LAVC) threshold. If all analyte input values are less than the LAVC, then a negative (NEG) result is returned in step 20. Otherwise the result is positive. For example, in one aspect, the LAVC is set to 30 in a scale of 0-100, however other LAVC thresholds may be used as desired.

In step 25, the kNN rows that will form the basis of a diagnosis are determined. For example, in one embodiment, the k nearest rows are determined by comparing the input analyte values with all rows in the database using a distance metric. In one aspect, the distance metric is a simple, N-dimensional distance metric of the form [(x₁−x₂)²+(y₁−y₂)²+ . . . +(N₁−N₂)²]^{1/2}, as described above. Thus, the k rows having the “shortest” distance from the input data are returned. In one embodiment, a specificity level threshold is used to judge whether a row can be included in the kNN rows that will form the basis of a diagnosis. In this embodiment, a row with a row concordance lower than the specificity level is not included. Row concordance generally refers to how closely a row from the kNN database table matched the patient input analyte values. In one aspect, the distance metric is used for row concordance. In one aspect, the process creates a row concordance value for each row, by comparing the patient's analyte values against the database table rows. The process then selects those rows that have row concordance values which meet the specificity level threshold. The process returns the rows with the best row concordance values. As many as k rows can be returned, but it is possible that fewer than k rows would meet the specificity level threshold. In one aspect, there are three adjustable specificity levels (although more or fewer may be used), a specific example of which follows:

10% (moderate);

35% (high); and

60% (highest).

Thus, if the specificity level is set at high and a row concordance value for a kNN row is determined to be lower than the “high” specificity threshold (e.g., <35%), that row is not used.

In step 30, the process determines whether the number of kNN rows returned or “created” in step 25 exceeds a minimum cutoff (MinCutoff) threshold. If fewer than “MinCutoff” rows were “created”, then “NA” or “no disease” is returned in step 35. The “NA” result refers to situations where the analytes passed the LAVC threshold (and therefore must be positive), but the algorithm (kNN) could not sufficiently associate them with a specific disease. Two such situations include:

Negative Special Case. kNN decided that the result was negative, but this would be inconsistent with the LAVC policy.

No diseases returned. The kNN could not (a) find enough good kNN rows to use in its calculations, or (b) identify one disease result with sufficiently strong disease concordance.

The kNN algorithm returns k number of rows, but only “creates” those rows where the row results meet the specificity level threshold. For example, suppose k=5, but 3 of the 5 returned rows have results that don't meet the specificity level threshold, thus only 2 rows are created. If MinCutoff is set at 3, “NA” would be returned. If the number of kNN rows is greater than MinCutoff, disease concordances are determined using the kNN rows in step 40. A disease concordance calculation refers to a calculation showing the importance of a disease result in the kNN Rows. In one aspect, the disease concordance for a particular disease identified by the kNN rows is equal to the number of disease rows divided by the number of total rows. For example, if k=5, and the rows indicate SS, SS, SS, SLE and MYO, then the disease concordance for SS=60.0%, the disease concordance for SLE=20% and the disease concordance for MYO=20%.

In step 45, the process determines whether a disease concordance is greater than a user-adjustable threshold, e.g., a very strong cutoff (VSC) threshold. For example, VSC may be set at 75% or 90%. If one disease concordance is greater than the VSC, a Negative Special Case (NSC) test is performed in step 50, wherein if it is determined that the disease name is Negative or NEG, the result is changed to “NA” (see above with respect to the Negative Special case). Otherwise, the disease name is output with an indication of a very strong (disease) concordance or association.

If one disease concordance is not determined to be greater than or equal to the VSC, then, in step 60 (FIG. 4), the algorithm determines whether the best disease concordance exceeds a user-adjustable moderate cutoff (MC) threshold value. For example, MC may be set at 25% or 30%. If the disease with the best concordance does not satisfy the MC threshold, a “NA” result is returned in step 65. If the disease with the best concordance satisfies the MC threshold, then, in step 70 the algorithm determines whether the disease with the second best concordance satisfies the MC threshold. If so, the NSC test is performed in step 75, and if the NSC test is satisfied, the best and second best diseases are returned in step 80 with an indication of their concordances. If the MC threshold is not satisfied, the NSC test is performed in step 85 for the best disease and, if satisfied, the best disease is returned in step 90 with an indication of its concordance. In the above example, where the concordance values for SS, SLE and MYO are 60%, 20% and 20%, respectively, if VSC is set at 65% and MC is set at 35%, the algorithm would return “SS with moderate concordance”.

In one aspect, a strong cutoff (SC) threshold is also used in the process. For example, the process may determine whether a disease satisfying the MC threshold also satisfies the SC threshold, and if so will provide an indication of strong concordance when outputting results. For example, if SC is set at 50% in the above example, the result would be “SS with strong concordance”. It is preferred that SC be set at 50% or higher.

Table 1 below shows an example of actual raw data used during a kNN match. Shown in Table 1 are results for eleven analytes, shown in AI (except analyte dsDNA which is in IU/ml), for the “unknown” as well as the 11 neighbors that were deemed the “best matches” by the process. In one aspect, to provide the data in Table 1 as a user display output, the AI values are preferably normalized as discussed above with referenvce to step 10 of FIG. 3. The dsDNA values would be converted by first dividing by 10 to convert to AI.

TABLE 1
Example of MDSS result

| | dsDNA | Chromatin | Ribosomal P | Sm | SmRNP | RNP | Scl-70 | Jo-1 | SSA | SSB | Centromere | Physicians' |
|---|---|---|---|---|---|---|---|---|---|---|---|---|
| Patient ID | (IUmL) | A.I. | A.I. | A.I. | A.I. | A.I. | A.I. | A.I. | A.I. | A.I. | A.I. | Diagnoses |
| Unknown | 62 | >8.0 | 3.4 | 0.3 | 0.4 | 0.8 | 0.8 | <0.2 | <0.3 | <0.4 | <0.5 | |
| Match #1 | 22 | >8.0 | 3.4 | 0.6 | 0.3 | 0.2 | <0.2 | <0.2 | 1 | <0.2 | <0.2 | SLE |
| Match #2 | 131 | 6.0 | >8.0 | 0.5 | 1.3 | 0.3 | <0.2 | <0.2 | 0.5 | <0.2 | <0.2 | SLE |
| Match #3 | 50 | >8.0 | 0.2 | 0.2 | 0.3 | <0.2 | 0.8 | <0.2 | <0.2 | <0.2 | <0.2 | SLE |
| Match #4 | 44 | 7 | <0.2 | <0.2 | 0.2 | <0.2 | 0.4 | <0.2 | <0.2 | <0.2 | <0.2 | SLE |
| Match #5 | 40 | 7.1 | <0.2 | <0.2 | <0.2 | 1.2 | <0.2 | <0.2 | <0.2 | <0.2 | <0.2 | NEG |
| Match #6 | >400 | >8.0 | <0.2 | 0.3 | <0.2 | 0.2 | <0.2 | <0.2 | <0.2 | <0.2 | <0.2 | SLE |
| Match #7 | 14 | >8.0 | 0.6 | 0.3 | 0.4 | 0.5 | <0.2 | <0.2 | <0.2 | <0.2 | <0.2 | SLE |
| Match #8 | 36 | >8.0 | <0.2 | <0.2 | <0.2 | 0.4 | <0.2 | <0.2 | <0.2 | <0.2 | <0.2 | SLE |
| Match #9 | 55 | 1.3 | 2.4 | <0.2 | <0.2 | 0.2 | <0.2 | <0.2 | <0.2 | <0.2 | <0.2 | SLE |
| Match #10 | 43 | 7.7 | <0.2 | 0.2 | <0.2 | 1.9 | <0.2 | <0.2 | <0.2 | <0.2 | 0.3 | NEG |
| Match #11 | 40 | 6.2 | <0.2 | <0.2 | <0.2 | <0.2 | <0.2 | <0.2 | <0.2 | <0.2 | <0.2 | SLE |

Algorithm selected match is SLE, a very strong association
AI ≧ 1.0 is Positive
dsDNA: ≧10 IU/ml is Positive, <5 is Negative, 5-9 is Indeterminate.

In this example, 9 of 11 of the “neighbors” had a diagnosis of SLE. Two did not; the physicians' diagnosis is technically speaking “Negative for a reportable CTD”. The actual diagnoses for these two samples supplied by the physicians were “Rheumatoid Arthritis” (RA) and “Disease-Free”. It may be surprising that two samples with dsDNA and Chromatin antibody levels so typical of SLE did not have this diagnosis, but this is a common occurrence. The patient with the RA diagnosis may have a condition called “RUPUS”, which is essentially an overlap disease of SLE and RA, seen in<5% of RA patients. The “disease free” patient may not meet four criteria for SLE, even though the serology results are strongly suggestive of this disease.

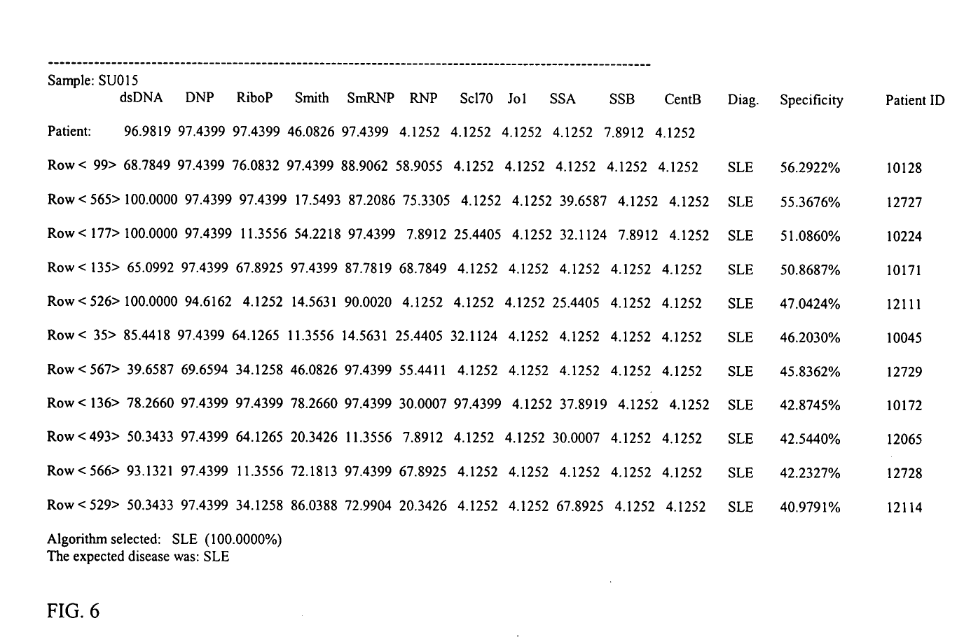

FIG. 6 illustrates a possible user display output generated for a KNN match. As shown, all values are normalized. “Specificity” refers to the row concordance value as discussed above. In this example, the MDSS reports a suggested diagnosis of SLE with a very strong (e.g., 100%) disease concordance.

Samples Used for the Training Set

The serum bank used for the library in the system of the present invention should meet a number of criteria. First, it should reflect the clinical diagnoses of highly qualified physicians, who follow recognized criteria. For the 1413 samples used in the training set example shown in Table 2, those with CTDs were collected by physicians at well recognized academic institutions: Oklahoma Medical Research Foundation, Stanford University and UCLA. These physicians also contributed samples from disease-free persons and samples from individuals with Fibromyalgia, osteoarthritis, and other connective tissue diseases such as Anti-Phospholipid Syndrome and Vasculitis. Additional samples were obtained from the Foundation for Blood Research in Maine; these samples were added so that the library would include samples with borderline and positive ANA's without a diagnosed CTD, since such samples are commonly encountered in the clinic. The actual distribution of samples in the training set is shown below in Table 2:

TABLE 2
Samples in the MDSS Training Set

| Physicians' diagnoses | N |
|---|---|
| CREST | 34 |
| CREST/SS | 1 |
| CTD | 29 |
| DMYO | 20 |
| DMYO/SLE | 1 |
| Fibromyalgia | 68 |
| Fibromyalgia/OA | 2 |
| Fibromyalgia/SLE | 1 |
| MCDT/SLE | 1 |
| MCTD | 25 |
| MCTD/CREST/SLE | 1 |
| MCTD/Raynauds/SLE | 1 |
| MCTD/SLE | 1 |
| NEG | 373 |
| OA | 70 |
| OA/Fibromyalgia | 2 |
| OA/RA | 3 |
| OA/SLE | 1 |
| PMYO | 51 |
| RA | 232 |
| RA/Fibromyalgia | 2 |
| RA/SS | 2 |
| Raynauds | 2 |
| SLE | 374 |
| SLE/CREST | 1 |
| SLE/Raynauds | 14 |
| SLE/SS | 19 |
| SLE/SS/Raynauds | 2 |
| SLE/UCTD | 1 |
| SS | 24 |
| SS/UCTD | 1 |
| Scleroderma | 29 |
| UCTD | 25 |
| Total | 1413 |

When the system generates a positive result, associations or suggestions for possible follow-up testing may be provided by a clinician based on the output as shown in Table 3.

TABLE 3
List of possible outputs

| System Comment | Appropriate follow-up testing |
|---|---|
| SLE | dsDNA, Chromatin, Sm, RiboP, |
| | Centromere |
| MCTD | RNP, SmRNP |
| Sjogren's Syndrome (SS) | SSA, SSB |
| Scleroderma/CREST/Raynaud's | Scl-70, Centromere |
| (SclCR) | |
| Myositis (MYO) | Jo-1 |
| NA (No Association - antibody | No pattern can be established based |
| levels do not show a pattern | upon the antibody pattern observed. |
| which can be associated with a | It is possible that the sample has |
| systemic Autoimmune disease.) | an atypical pattern. No further testing is |
| | indicated unless there is strong |
| | clinical evidence supporting one of |
| | the above diseases |

The present invention is particularly useful for improving ANA screens. The ANA screen is useful as a “rule out” test, especially for SLE, but has very little value in differential diagnosis. As a follow-up to a positive ANA screen, the physician must request determinations of specific antibodies. The system of the present invention can assist the physician with this task by offering associations or suggestions to consider the appropriate follow-up testing. Examples are shown in Table 3. These associations are similar in nature to the traditional association of “homogeneous” pattern with SLE and “nucleolar pattern” with Scleroderma; physicians have used these traditional associations for decades. Their high level of comfort with these traditional associations has made the transition from IFA methods to the newer EIA methods difficult for many laboratories. With the addition of the system of the present invention to the ANA screen, the ability of a laboratory to direct the physician towards the appropriate follow-up test is restored and in fact improved.

The NA Association and Reports Showing More Than One Association

As was mentioned above and in Table 3, one of the possible outputs of the system includes “NA (No Association—antibody levels do not show a pattern which can be associated with a systemic Autoimmune disease).” In one aspect, this output indicates that at least one positive antibody was detected, but that there are insufficient matches against members of the library that exceed a given cutoff, and/or that any disease match represents less than a certain percentage, e.g., 25%, of the total. Because there may be many samples from non-CTDs in the library, such as fibromyalgia and RA, the NA association often indicates that the matches obtained were for RA rather than for SLE.

Many of the samples in the library may contain no positive antibodies. Such samples are preferably included in the library because they allow for the system to calculate specificity if required. The majority of the samples from fibromyalgia, osteroarthritis (OA), Dermatomyositis (DMYO) and Rheumatoid Arthritis patients are antibody negative.

One of the principal advantages of using a kNN algorithm is that it allows two associations to be reported. As Table 2 indicates, physicians often establish more than one diagnosis; the combination of SLE and SS (Sjogren's disease) is the most common. Many patients with SLE often have Raynaud's syndrome as well, which is associated with anti-Centromere and anti-RNP antibodies. The system of the present invention will frequently report SLE/SS, SLE/SclCR and SLE/NA.

The example shown in Table 1 is from a patient (“unknown”) with very elevated anti-dsDNA and anti-Chromatin. The presence of two very elevated antibodies almost always leads to a good disease association. However, many samples lack such a definitive pattern. In such cases, a typical output association might be SLE/NA, indicating the resemblance to SLE patients but also resemblance to patients with other diseases not reported by the system, as defined in Table 3.

Because one goal of the present system is to provide a high predictive value, samples with SLE will sometimes be reported as NA. These samples may contain diagnostic antibodies such as anti-dsDNA and anti-Sm at very modest levels.

As Table 1 indicates, certain patterns may be strongly associated with a disease, while others may have a more modest association. SLE/NA represents the most modest association of a given unknown sample with Lupus. In one aspect, samples with stronger associations are identified in the output as “moderate,” “strong” or “very strong” associations. Table 1 shows an example of a very strong association (e.g., greater than 75% of the chosen matches have the same diagnosis). Additionally, FIG. 5 illustrates an example of a display pane including a legend indicating strength of disease associations.

Screening Output Versus Detailed Antibody Report

In the USA, physicians are expected to follow clinical practice guidelines in choosing patients for ANA testing and for ordering follow-up tests. Since specific antibody tests are not eligible for panel reimbursement, most hospitals and institutions may not be able to obtain the antibody outputs, e.g., 11 antibody outputs, produced by the present system, as shown in Table 1. By combining the system output with a “Positive” output of the ANA screen, in one aspect, the present invention offers the physician a report option that provides the disease associations without incurring the additional expense of ordering specific antibodies. The sample used to generate the screening result generally cannot be used to generate specific antibody results; the laboratory must re-analyze the specimens with an appropriate order.

In some hospitals, clinical practice guidelines may support the direct report of all antibody outputs, either as a first-line test or as a follow-up to a screening test performed by another method. Thus in one aspect, the system of the present invention is advantageously configurable to provide a report option that combines the antibody outputs with the associations. FIG. 5 illustrates an example of such an output represented in a display pane. In one aspect, no antibody output is provided for any sample unless all, e.g., eleven, specific antibodies are requested.

Additional Useful Intelligent Systems/Statistical Analysis Methods

A Bayesian algorithm differs from the k-nearest neighbor algorithm by calculating results in terms of probabilities based on all of the data in the training set, rather than a limited group selected on the basis of its proximity to the test patient. A Bayesian algorithm extracts two types of data from the data points in the training set database, one representing the disease prevalence and the other representing statistical measurements such as mean and standard deviation. A probability density is determined for the patient sample and each disease, and the likelihood of each disease is calculated. The relative value of each disease is then determined from a ratio of the likelihood for that disease divided by the total of the likelihoods for all of the diseases, and the diagnosis is achieved by selecting the disease with the highest relative value. Like the k-nearest neighbor algorithm, Bayesian algorithms can be used to detect the presence of two diseases in a single patient.

The neural network system is a network of nodes or simple numerical processing units that are arranged in layers and connected by communication channels. The channels are weighted differently in accordance with information imported from the training set. Data are passed from the first layer to successive layers in accordance with the different weights assigned to the channels. Each node in a given layer multiplies all the node values from the previous layer by the weights of the connection channels that join the nodes of the two layers, and then determines the sum of these values. The sum is then passed through a sigmoid (“S-curve”) function to identify the disease.

Principal component analysis is a multivariate technique for reducing matrices of data to their lowest dimensionality by use of orthogonal factor space. According to this technique, the training set is processed to identify the number of principal components. This information is then used to model the patent test data using such techniques as target transformations or curve fitting. Neither the principal component nor the neural network analyses are suitable for the detection of two diseases.

Other pattern recognition techniques that are known to those skilled in statistical data analysis can be used as well, and their adaptation to the systemic autoimmune diseases addressed by this invention will be readily apparent to the skilled artisan. Regardless of the pattern recognition technique that is used, the technique and its application in this context are readily susceptible to computerized implementation. Software specifically designed for particular analyses is readily developed and a routine matter to the skilled software engineer.

Antibodies and Assays

The test data used in the present invention include values that are proportional to, or otherwise representative of, the levels of various antibodies that are associated to various degrees with systemic autoimmune diseases. Currently, over 100 antibodies are known to be expressed in autoimmune diseases. Examples are listed in Peter, J. B., et al., Autoantibodies, Elsevier Science B.V., Amsterdam (1996), the contents of which are incorporated herein by reference. The antigens to many of these antibodies are commercially available, while the antigens to others are readily synthesized based on descriptions of them that are available in the literature. Some of the sources of these antigens are BiosPacific, of Emeryville, Calif., USA; Immunovision, of Springdale, Ark., USA; and KMI Diagnostics, Inc., of Minneapolis, Minn., USA. Examples of the antibodies that are expressed in autoimmune diseases, identified by the antigens to which they bind in an immunoassay, are listed below:

SSA 60

SSA 60

SSA 52

SSB 48

Sm BB′

Sm D1

Sm

SMRNP

RNP 68

RNP A

RNP C

Fibrillarin

Riboproteins P0, P1, and P2

dsDNA

Nucleosome

Ku

Centromere A

Centromere B

Scl-70

Pm-Scl

RNA-Polymerases 1, 2, and 3

Th

Jo-1

Mi-2

PL7

PL12

SRP

The present invention is not intended to be limited to antibodies identified by the antigens in the above list. Instead, the number of antibodies that are detected and quantitated from the training set, and the number of antibodies detected and quantitated in the patient sample as well, may vary and are not critical to this invention. The particular antibodies selected and the number of such antibodies in the selected group may however affect the accuracy of the assay and its ability to identify the systemic autoimmune disease(s) present. In most cases, it is contemplated that from 10 to 100 antibodies will be used, preferably from 15 to 25. In preferred embodiments of the invention, at least 10 of the antibodies identified by the antigens in the above list are used, and in further preferred embodiments, all of the antibodies in the above list are used.

For example, in one embodiment, the following antibodies (identified by the antigens to which they bind) are used for a test set:

SSA 60,

SSA 52,

SSB 48,

Sm,

SMRNP

RNP 68,

RNP A,

Riboproteins P0, P1, and P2,

dsDNA,

Nucleosome,

Centromere B,

Scl-70, and

Jo-1.

However, for the list of antigens above, it may not be possible to identify dermatomyositis.

In one embodiment, an antigen called SMRNP is tested for and used. This is a multi-protein complex that includes both SmD1 and RNP68 RNP A, and can be used as a way of “verifying” the results for the individual antigens as it helps the system sort out these proteins.

Similarly, the number of reference samples of different sources used to develop the training set may vary as well and is not critical to this invention, although the number selected may affect the range of autoimmune diseases that can be detected. In most cases, it is contemplated that 100 to 10,000 reference samples will be used, preferably from 200 to 2000 or more.

The quantitative levels of each antibody are determinable by conventional antibody assays. Immunoassays are generally preferred. Both antigen capture (indirect immunoassays) and class capture assays can be used, antigen capture being preferred, and detection can be achieved by enzyme labels, radioactive labels, or fluorescent labels. The assays may be colorimetric, luminometric, fluorometric, enzymetric, radiometric, or other methods such as nephelometry, turbidimetry, particle counting, or visual assessment. Examples of enzyme labels are alkaline phosphatase, -D-galactosidase, glucose-6-phosphate dehydrogenase, horseradish peroxidase, -lactamase, melittin, and urease. Examples of radioisotopic labels are cobalt-57 and iodine-125. Examples of fluorescent labels are succinimidyl esters and vinyl sulfones of xanthenes, cyanines, coumarins, benzimides, phenanthridines, ethidium dyes, acridine dyes, carbazole dyes, phenoxazine dyes, porphyrin dyes, quinoline dyes, and naturally occurring protein dyes. Fluoresceins and rhodamines are particular types of xanthene dyes. Specific examples are 6-carboxyfluorescein, 6-carboxy-4′,5′-dichloro-2′,7′-dimethoxyfluorescein, N,N,N′,N′-tetramethyl-6-carboxyrhodamine, 6-carboxy-X-rhodamine, 5-carboxyrhodamine-6G, 5-carboxyrhodamine-6G, tetramethylrhodamine, Rhodamine Green, and Rhodamine Red. Umbelliferone is an example of a coumarin. Hoechst 33258 is an example of a benzimide dye. Texas Red is an example of a phenanthridine dye. Examples of cyanine succinimidyl ester dyes are sulfoindocyanine succinimidyl esters, (carboxyalkyl)cyanines succinimidyl esters, and BODIPY succinimidyl esters (Molecular Probes, Inc., Eugene, Oreg., USA). Examples of naturally occurring protein dyes are phycoerythrins. Many other examples will be readily apparent to those skilled in the art.

The sequence and manner of performing quantitative assays of multiple antibodies in the practice of this invention may vary widely. In preferred embodiments, the assays are performed simultaneously in a single sample by a multiplexed system that differentiates the assays from each other and thus provides individual values for each of the antibodies. This may be achieved in a variety of ways. Heterogeneous binding assays, in which one of the binding members is immobilized on a solid phase, are the most efficient, since they provide a ready means for separating the bound pairs from unbound species. Multiplexed heterogeneous binding assays in which solid particles, preferably magnetic microbeads, are used as the solid phase, are illustrative examples. In these assays, the beads are sorted into groups, each group containing the reagents for a single assay covalently bonded to the bead surface, the various groups differentiable from one another by virtue of distinguishing characteristics that permit separate detection of the assay result in one group from those in the other groups. The differentiating parameter may be particle size, fluorescence (independent of the fluorescent label used in the assay, when fluorescent assay labels are in fact used), light scatter, light emission, or absorbance. The binding member that is covalently bonded to the bead surface will vary depending on the type of immunoassay that is being used. Since the analytes in accordance with this invention are all antibodies, the antigens by which the antibodies are defined serve as particularly convenient binding members bonded to the bead surface. Actual differentiation of the bead groups is readily achieved by flow cytometry.

When magnetic beads are used, the magnetic character permits quick separation of the solid and liquid phases and convenient washing of the solid phase. A magnetic character can be imparted to the beads by using beads made of paramagnetic materials, ferromagnetic materials, ferrimagnetic materials, or metamagnetic materials. The magnetically responsive material is preferably only one component of the bead, the remainder consisting of a polymeric material to which the magnetically responsive material is affixed or otherwise combined. The polymeric material can be chemically derivatized to permit attachment of the antigen or other assay reagent that enters into the binding reaction.

Multiplex systems of this description as well as others useful in the practice of this invention are disclosed in pending U.S. patent application Ser. No. 09/302,920, filed Apr. 30, 1999, entitled “Multiplex Flow Assays Preferably With Magnetic Particles as Solid Phase,” Michael I. Watkins et al., inventors. The entire disclosure of application Ser. No. 09/302,920 is incorporated herein by reference.

The biological samples on which the tests are performed can be any biological fluid extracted from the patient that is likely to have a detectable antibody population that is characteristic of the disease(s) sought to be investigated. Serum, plasma, urine, or cerebrospinal fluid may be used. Serum samples are preferred.

This invention is useful in the detection and diagnosis of systemic autoimmune diseases in general. Examples include systemic lupus erythmatosus (SLE), Sjögren's syndrome (SS), scleroderma, polymyositis and dermatomyositis, CREST (calcinosis, Raynaud's phenomenon, esophageal involvement, sclerodactyly, and telangiectasis), mixed connective tissue disease, Wegener's disease, rheumatoid arthritis, and spondylarthropathies.

The foregoing is offered primarily for purposes of illustration. Further modifications and variations of the various parameters of the composition and method of this invention will be readily apparent to those skilled in the art. For example, the pattern recognition algorithm can be adjusted to control the rate of false negative results, by setting a boundary that differentiates “healthy” from “unhealthy” for a particular disease at a higher level than the corresponding boundary for other diseases, thereby lowering the number of samples that will indicate the presence of the disease. These and other variations are included within the scope of the invention.

What is claimed is:

1. A computer-implemented method of identifying whether a patient test sample is associated with one or more of a plurality of specific systemic autoimmune diseases (SADs) based on autoantibody levels present in the patient test sample; the method comprising:
storing a plurality of reference data sets in a memory, each data set having values representing levels for each of a plurality of specific autoantibodies, wherein said reference data sets include, for each of said specific SADs, at least one reference data set having an association with the specific SAD, and wherein said reference data sets include at least one reference data set associated with none of the specific SADs;
receiving a sample data set having values representing levels for each of said plurality of autoantibodies for a patient test sample; and
automatically applying a k-nearest neighbor process to the sample data set and the reference data sets to produce a statistically derived decision indicating whether the patient test sample is associated with none, one or more of said specific SADs.

2. The computer-implemented method of claim 1, wherein the SADs include two or more systemic autoimmune diseases selected from the group consisting of systemic lupus erythmatosus, scleroderma (SLE), Sjögren's syndrome (SS), polymyositis (PMYO), dermatomyositis (DMYO), CREST, and mixed connective tissue disease (MCTD).

3. The computer-implemented method of claim 1, wherein the SADs include two or more systemic autoimmune diseases selected from the group consisting of systemic lupus erythmatosus (SLE), scleroderma, Sjögren's syndrome (SS), myositis (MYO), polymyositis (PMYO), dermatomyositis (DMYO), CREST, connective tissue disease (CTD), fibromyalgia, osteroarthritis (OA), Reynaud's syndrome and Rheumatoid arthritis (RA).

4. The computer-implemented method of claim 1, wherein said plurality of autoantibodies comprises antibodies to at least ten of the following antigens:
SSA 60,
SSA 52,
SSB 48,
Sm BB′,
SmD1,
Sm,
SmRNP
RNP 68,
RNP A,
RNP C,
Fibrillarin,
Riboproteins P0, P1, and P2,
dsDNA,
Nucleosome,
Ku,
Centromere A,
Centromere B,
Scl-70,
Pm-Scl,
RNA-Polymerases 1, 2, and 3,
Th,
Jo-1,
Mi-2,
PL7,
PL12, and
SRP.

5. The computer-implemented method of claim 1, wherein said plurality of autoantibodies consists of antibodies to the following antigens:
SSA 60,
SSA 52,
SSB 48,
Sm,
SMRNP,
RNP 68,
RNP A,
Riboproteins P0, P1, and P2,
dsDNA,
Nucleosome,
Centromere B,
Scl-70, and
Jo-1.

6. The computer-implemented method of claim 1, further including generating a display output including said indication of whether the patient test sample is associated with none, one or more of the specific SADs.

7. The computer-implemented method of claim 6, wherein generating includes transmitting display output data to a remote computer system and rendering the display output on a display screen coupled with the remote computer system.

8. The computer-implemented method of claim 1, wherein receiving includes receiving the sample data set from an automated test system over a network connection.

9. The computer-implemented method of claim 8, wherein storing includes receiving the reference data sets from the automated test system over the network connection.

10. The computer-implemented method of claim 1, wherein storing includes receiving the reference data sets from one or more test sources.

11. The computer-implemented method of claim 1, wherein the k-nearest neighbor process includes determining, for each of the reference data sets, a concordance value between the sample data set and the reference data set, and comparing each concordance value to a threshold value, wherein only a first plurality of the reference data sets having a concordance value that exceeds the threshold value are used by the process.

12. The computer-implemented method of claim 11, wherein the k-nearest neighbor process further includes determining, for each of the reference data sets, a distance metric value between the sample data set and the reference data set.

13. The computer-implemented method of claim 11, wherein the process further includes:
determining whether the number of the first plurality of reference data sets exceeds a minimum cutoff value, and
if not, providing an indication that the patient test sample is associated with none of the specific SADs, and
if so, determining whether the patient test sample is associated with one or more of the specific SADs.

14. The computer-implemented method of claim 11, wherein the process further includes determining a disease concordance value for each of the first plurality of reference data sets.

15. The computer-implemented method of claim 14, wherein determining a disease concordance value includes:
for each SAD associated with the first plurality of reference data sets:
adding the number of the first plurality of reference data sets associated with that SAD and dividing by the total number of the first plurality of reference data sets to produce a disease concordance value for that SAD.

16. The computer-implemented method of claim 15, further including comparing each disease concordance value with a first threshold value, and returning the SAD associated with the concordance value that exceeds the first threshold value.

17. The computer-implemented method of claim 16, further including comparing each disease concordance value with a second threshold value, and returning the SAD associated with the concordance value that exceeds the second threshold value.

18. A computer system configured to provide output data indicating whether a patient test sample is associated with one or more of a plurality of specific systemic autoimmune diseases (SADs) based on autoantibody levels present in the patient test sample; the system comprising:
storage means for storing a plurality of reference data sets, each data set having values representing levels for each of a plurality of specific autoantibodies, wherein said reference data sets include, for each of said specific SADs, at least one reference data set having an association with the specific SAD, and wherein said reference data sets include at least one reference data set associated with none of the specific SADs;
a means for receiving a sample data set having values representing levels for each of said plurality of autoantibodies for a patient test sample;
a means for processing the sample data set and the reference data sets using a k-nearest neighbor process to produce a statistically derived decision indicating whether the patient test sample is associated with none, one or more of said specific SADs; and
a means for providing output data including the statistically derived decision.

19. The system of claim 18, wherein the SADs include two or more systemic autoimmune diseases selected from the group consisting of systemic lupus erythmatosus (SLE), scleroderma, Sjögren's syndrome (SS), myositis (MYO), polymyositis (PMYO), dermatomyositis (DMYO), CREST, connective tissue disease (CTD), fibromyalgia, osteroarthritis (OA), Reynaud's syndrome and Rheumatoid arthritis (RA).

20. The system of claim 18, wherein said plurality of autoantibodies comprises antibodies to at least ten of the following antigens:
SSA 60,
SSA 52,
SSB 48,
Sm BB′,
SmD1,
Sm,
SmRNP,
RNP 68,
RNP A,
RNP C,
Fibrillarin,
Riboproteins P0, P1, and P2,
dsDNA,
Nucleosome,
Ku,
Centromere A,
Centromere B,
Scl-70,
Pm-Scl,
RNA-Polymerases 1, 2, and 3,
Th,
Jo-1,
Mi-2,
PL7,
PL12, and
SRP.

21. The system of claim 18, wherein said plurality of autoantibodies consists of antibodies to the following antigens:
SSA 60,
SSA 52,
SSB 48,
Sm,
SmRNP,
RNP 68,
RNP A,
Riboproteins P0, P1, and P2,
dsDNA,
Nucleosome,
Centromere B,
Scl-70, and
Jo-1.

22. The system of claim 18, wherein the means for providing the output data includes one of a monitor for displaying the output data, a printer for printing the output data and a communication interface device for providing the output data to a separate computer system.

23. The system of claim 18, wherein the means for receiving the sample data set includes one of an interface device configured to receive data from a remote automated test system, a manual input device, and a device configured to read data from a computer readable medium.

24. The system of claim 18, wherein the storage means includes one of a RAM, a ROM, a computer readable disk medium, a hard disk drive and a separate database system.

25. The system of claim 18, wherein the k-nearest neighbor process determines, for each of the reference data sets, a concordance value between the sample data set and the reference data set, and compares each concordance value to a threshold value, wherein only a first plurality of the reference data sets having a concordance value that exceeds the threshold value are used by the process.

26. The system of claim 25, wherein the k-nearest neighbor process further determines, for each of the reference data sets, a distance metric value between the sample data set and the reference data set.

27. The system of claim 25, wherein the k-nearest neighbor process further determines whether the number of the first plurality of reference data sets exceeds a minimum cutoff value, and
if not, provides an indication that the patient test sample is associated with none of the specific SADs, and
if so, determines whether the patient test sample is associated with one or more of the specific SADs.

28. The system of claim 25, wherein the k-nearest neighbor process further determines a disease concordance value for each of the first plurality of reference data sets.

29. The system of claim 28, wherein a disease concordance value is determined for each SAD associated with the first plurality of reference data sets by adding the number of the first plurality of reference data sets associated with that SAD and dividing by the total number of the first plurality of reference data sets to produce a disease concordance value for that SAD.

30. The system of claim 29, wherein the process further compares each disease concordance value with a first threshold value, and returns the SAD associated with the concordance value that exceeds the first threshold value.

31. The system of claim 30, wherein the process further compares each disease concordance value with a second threshold value, and returns the SAD associated with the concordance value that exceeds the second threshold value.
